(19) United States
(12) Patent Application Publication
(10) Pub. No.: US 2023/0397728 A1
(43) Pub. Date: Dec. 14, 2023

(54) ACTIVE CHAIR ACTIVITY TRACKING SYSTEM

(71) Applicant: Kinetic Furniture of Vermont Inc., Colchester, VT (US)

(72) Inventor: Turner Osler, Colchester, VT (US)

(21) Appl. No.: 18/456,726

(22) Filed: Aug. 28, 2023

Related U.S. Application Data
(63) Continuation-in-part of application No. 17/820,739, filed on Aug. 18, 2022.
(60) Provisional application No. 63/263,346, filed on Nov. 1, 2021.

(51) Int. Cl. A47C 3/02; G06F 3/01; A61B 5/00

(52) U.S. Cl. CPC A47C 3/02; G06F 3/011; A61B 5/486; A47C 15/004

(57) ABSTRACT

A user's motion while seated on an active chair is monitored and the user's metabolic expenditures stemming from the motion are determined. Information associated with or derived from the measured motion and/or determined metabolic expenditures may be be displayed. Motion sensors and gyroscopes are configured to detect the motion of the active chair associated with active sitting while the user is sitting. Data from these sensors is used to determine sitting activity of the user. In addition, a sitter's posture may be monitored and alerts sent to encourage the siter to improve posture. A training aspect may also be included that assists sitters/users to improve posture generally by guiding them through a patterned series of movement designed to improve pelvic mobility and awareness.

### FIELD OF THE INVENTION

The present invention relates to seating devices. In particular, the present invention provides systems and methods for tracking activity of a user of an ergonomic seat.

### BACKGROUND

Human bodies are built to move and generally require constant activity to remain supple and healthy. Unfortunately, modern life involves a good deal of sitting. In fact, many professions require many hours of simply sitting, which puts an unnatural demand on the human body-so unnatural that children instinctively rebel against it.

Sitting, and especially sitting still, aligns human bodies oddly, and denies joints the constant small adjustments that help to circulate the joint fluid which helps nourish the delicate cartilage lining of the joints. Additionally, sitting still denies core muscles the exercise involved in aligning and realigning our spines, exercise vital to keeping our core musculature strong and responsive. Moreover, extended and repetitive sitting has been linked to other health maladies. Indeed, the mismatch between our 21st-century-built environment and our hunter-gatherer-optimized bodies has led to a variety of serious health problems: obesity, diabetes, heart disease, and even cancer, a litany of acquired diseases that can culminate in early death. These effects are not subtle with a 50% increase in cancer risk among those who sat the most. Perhaps more importantly, simply reducing sitting could increase our lifespans by as much as two years.

One potential solution to these health issues may be the use of active chairs, which are designed to cause users to sit actively and thus may allow for the recapture of the healthy blood chemistry of our hunter-gatherer forebears. These chairs allow for movement while sitting, and so allow us to sit all day as our modern lives require without suffering the harm brought on by sitting inertly. An example of such an active chair is described in commonly owned U.S. Pat. No. 10,010,758.

A way to monitor or quantify the motion induced by sitting in such active chairs is to measure the motion.

### SUMMARY OF THE DISCLOSURE

A system for tracking sitting activity of a user includes an active chair with a seat, a rocking mechanism, and a base. A motion sensor is configured to detect motion of the seat about the rocking mechanism and a gyroscope is configured to determine a change in orientation of the seat. A processor is operatively connected with a memory, the motion sensor, and the gyroscope, and the memory stores computer-executable instructions for causing the processor to receive information from the motion sensor and the gyroscope, analyze, during a user sitting session, the information from the motion sensor and gyroscope to determine whether the seat has undergone motion indicative of active sitter motion, and determine, based on a total amount of motion indicative of active sitting motion during the user sitting session, a number of calories used by the user during the user sitting session.

A method for tracking active sitting includes obtaining motion information from a motion sensor configured to detect motion associated with active sitting motions of an active chair when a user is seated on the active chair, obtaining orientation information from a gyroscope configured to detect a change in orientation of a seat of the active chair when the user is seated on the active chair, identifying, based on the motion information and the orientation information, by a processor, whether the seat has undergone active sitter motion, determining a total amount of active sitting motion during an active sitting session, and determining a number of calories used based on the total amount of active sitting motion during the user sitting session.

In another embodiment, a system for tracking a sitter's posture includes an active sitting chair including a seat, a rocking mechanism, and a base, a sensor configured to detect a tilt of the seat with respect to a horizontal axis, and a device having a memory and a processor in communication with the sensor. The device is in communication with the sensor and the memory stores computer-executable instructions for causing the processor to receive data about the tilt of the seat from the sensor and determine a tilt value, compare the tilt value to a tilt threshold, and send an alert perceivable to a sitter on the active sitting chair when the tilt value is greater than the tilt threshold for a predetermined amount of time.

In addition, a method for tracking a sitter's posture is provided that includes receiving data from a sensor configured to detect a tilt with respect to a horizontal axis of a seat of an active sitting chair having a rocking mechanism, determining a tilt value from the data, comparing the tilt value to a tilt threshold, and sending an alert perceivable to a sitter on the active sitting chair when the tilt value is greater than the tilt threshold for a predetermined amount of time.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a perspective view of an active chair with an attached motion sensor in accordance with an embodiment of the present invention;

FIG. 2 is a flow diagram of a method of measuring physical activity of a sitting user in accordance with an embodiment of the present invention;

FIG. 3 is a flow diagram of a method of determining metabolic expenditure of a sitting user in accordance with an embodiment of the present invention;

FIG. 4 is an illustration of a display screen with active sitting data in accordance with an embodiment of the present invention;

FIG. 5 is a perspective view of an active chair with a user seated on the active chair wherein a motion sensor is held by and associated with the user in accordance with another embodiment of the present invention;

FIG. 6 is a flow diagram of a method of measuring physical activity of a sitting user having a motion sensor in accordance with an embodiment of the present invention; and

FIG. 7 is a flow diagram of another method of measuring physical activity of a sitting user having a motion sensor in accordance with an embodiment of the present invention;

FIG. 8 a flow diagram for measuring physical activity of a sitting user having a motion sensor in accordance with an embodiment of the present invention;

FIGS. 9A-9C are illustrations depicting a series of screenshots from a display screen displaying a target and a representation of seat position being updated in real time in accordance with another aspect of the invention;

FIG. 10 is a perspective view of an active chair with a user seated on the active chair wherein a sensor detects the tilt of the seat in accordance with another embodiment of the present invention; and

FIG. 11 is a flow diagram of a method for detecting the posture of a sitting user in accordance with an embodiment of the present invention.

### DESCRIPTION OF THE DISCLOSURE

The present invention provides for measuring a sitting user's motion associated with active sitting movements while seated on an active chair, determining the user's metabolic expenditures stemming from the sitting user's motion, and displaying information associated with or derived from the measured motion and/or determined metabolic expenditures. A sensor, such as an accelerometer and/or gyroscope, may be attached to an active sitting chair or attached to the sitter, such as by a strap or in a pocket of the sitter. The sensor is in communication with (or part of) an electronic device such as a smart phone. The motion detected by the sensor can be translated into data related to other forms of activity, such as calories burned. In addition, the detected motion can be used to create a real time position indicator on a display screen in which the position of the sensor is shown to move in sync with the sitter's motion on the active chair.

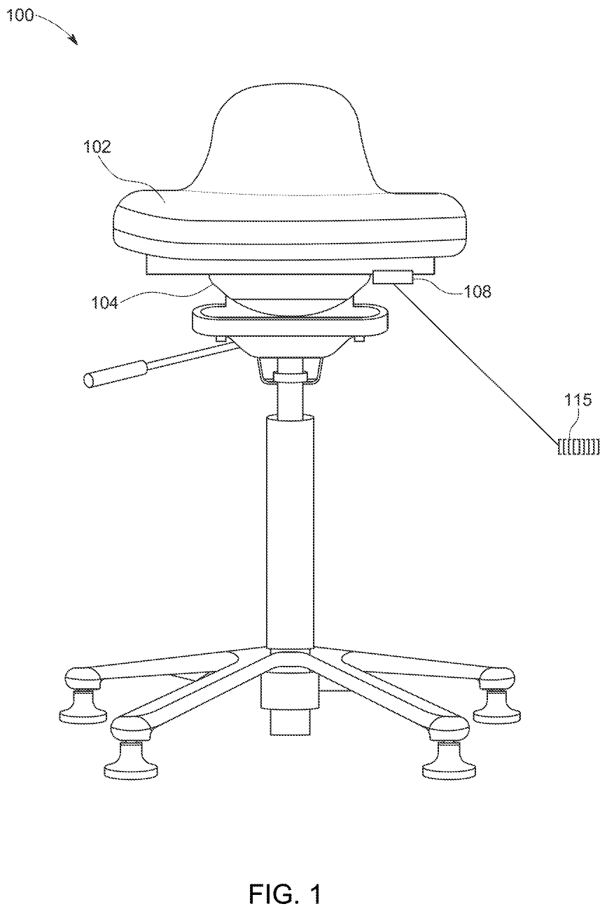

In an embodiment, an active sitting chair, such as active chair 100 shown in FIG. 1, includes a seat 102, a rocking or wobble mechanism 104 that allows for and encourages motion by a user sitting in chair 100, and one or more sensors 108 attached to chair 100. Sensors 108 may include an accelerometer and a gyroscope which may be attached at any appropriate location in order to detect acceleration and seat orientation along the X, Y, or Z axis (for example motion associated with the rocking and wobbling motion induced by wobble mechanism 104), such as beneath the seat as shown in FIG. 1. A 3-axis accelerometer could also be affixed to the person of the sitter, such as within a phone in a pocket of the sitter. Data from motion and orientation measured by sensors 108 may be processed by the accelerometer (e.g., if it is a smart sensor) or transmitted to a processor (preferably wirelessly) in an electronic device. Transmission may occur continuously, at intervals, or be collected after a period of sitting ends, and may be initiated by the sitter providing input that an active sitting session has begun.

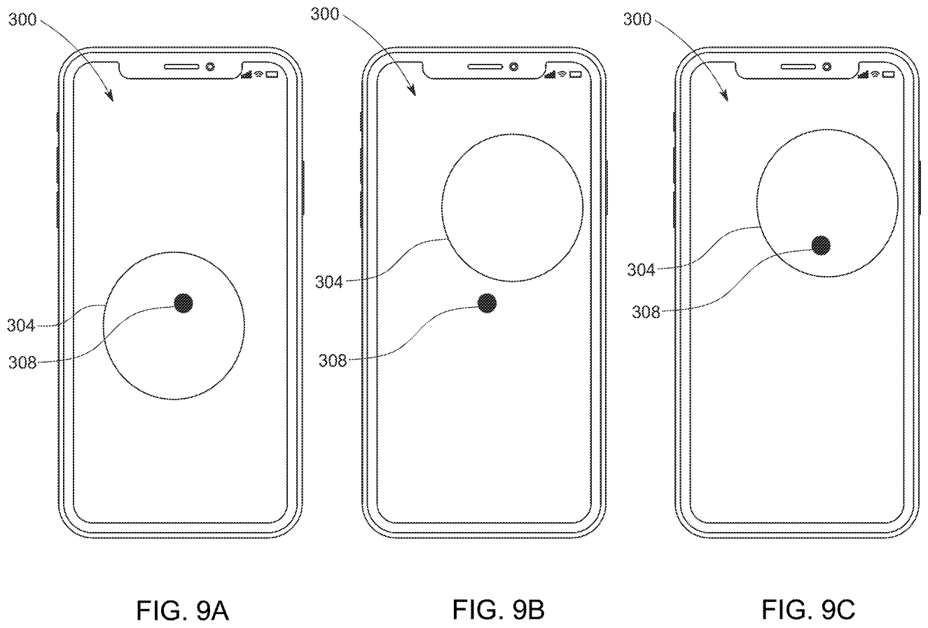

Motion data is used to determine a level of physical activity of the user sitting in chair 100. For example, data from sensors 108 is communicated to a processor 115 (shown schematically in FIG. 1) having a memory and may then be used to determine the frequency of motion, as well the amount of motion detected by the accelerometer along three axes, X, Y, and Z. This motion can be converted to calories burned by the sitter based on various parameters and associations, including the use of regression equations, for example, as well as the dimensions of chair 100 and the extent of motion enabled by wobble mechanism 104. In addition, tilt sensors gather data that can be used to display a representation of the movement of an active chair in real time on a display screen (as shown for example in FIGS. 9A-9C), allowing for the gamification of active sitting as a way to encourage users to move more, or to move in prespecified ways so as to learn new movement patterns.

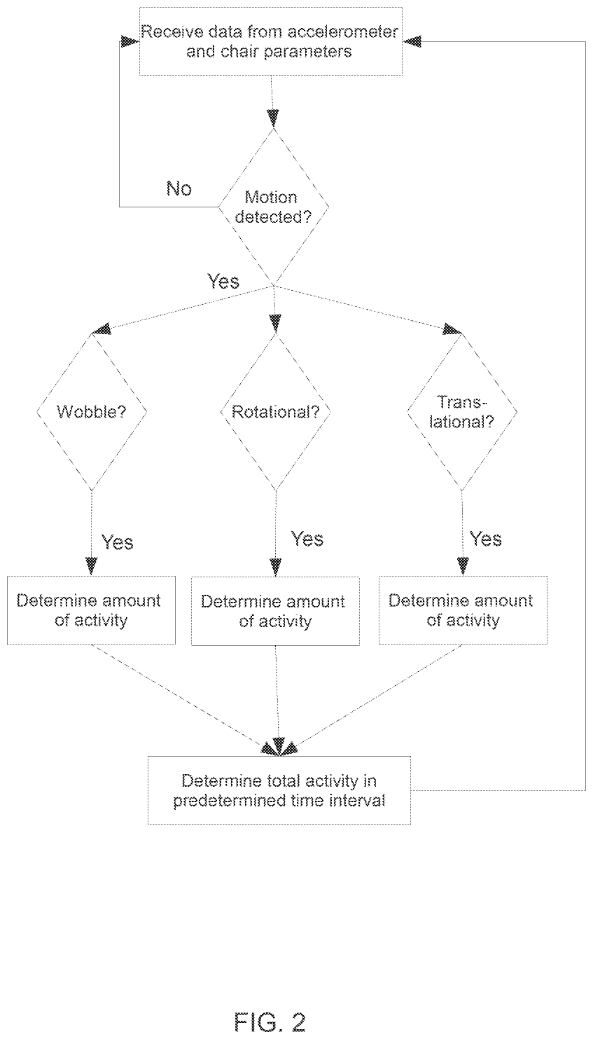

An exemplary process for determining activity level is outlined in FIG. 2. Data is received from the active chair accelerometer as well as the parameters for that active chair, such as seat size and wobble amount (e.g., tilt angle) allowed. It is then determined whether any motion was detected by the accelerometer. If no motion is detected, the process returns to the first step and waits to receive data from the accelerometer. If motion is detected, it may be converted to a measure of activity (e.g., counts). From those amounts of activity, the total amount of user sitting activity on the active chair that occurred during a predetermined time interval is determined. In addition, it may be determined whether the motion includes wobble, rotational, and/or translational motion, in which the amount of activity involved in creating such motion is determined based on the accelerometer data received along with the active chair parameters.

From the physical activity determined, metabolic expenditure of the user can be determined based further on information about the user, such as height, weight, age, etc. These parameters together with the measured chair activity levels can be used to estimate metabolic energy expenditures resulting from the active sitting. This may be based on calculations of energy required to perform such movements that result in the measured chair motion and/or measurements of physiologic parameters (such as respiration increases over baseline for similar individuals) that are correlated with the measured motion by the accelerometer.

From these increases in energy expenditure, i.e., caloric use increases compared to sitting in non-active chairs, may be determined for users as well as other outputs such as step equivalents or active chair sitting activity levels.

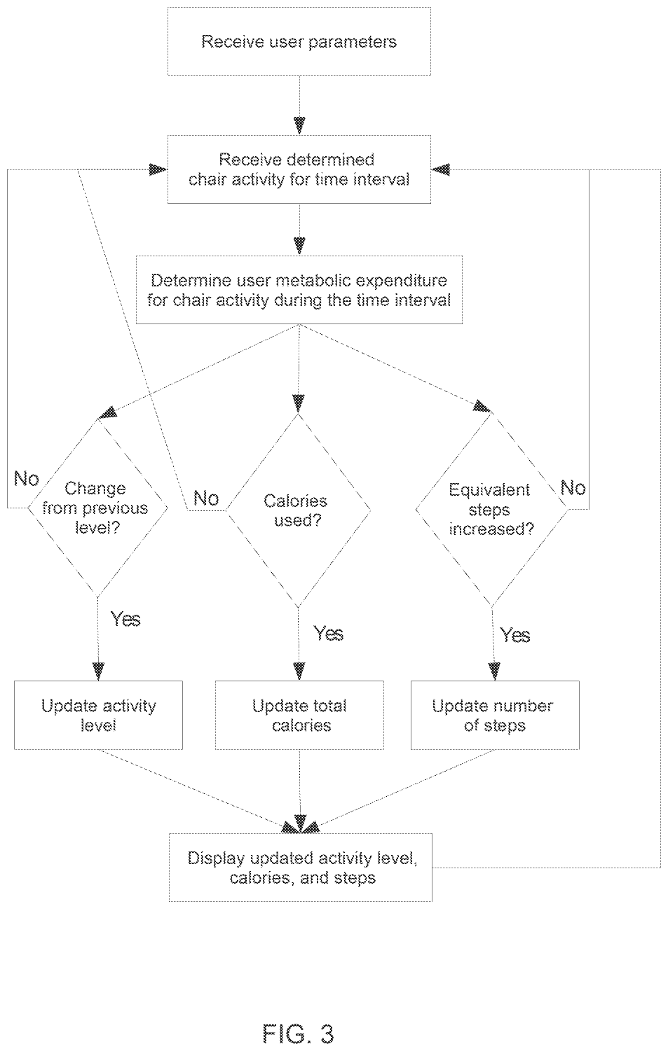

An exemplary process for determining user energy expenditure is outlined in FIG. 3. User parameters are received from input or other sources, and the total amount of user sitting activity on the active chair that occurred during a predetermined time interval is received. From the user parameters and the total amount of user sitting activity detected during an active sitting session, a user metabolic expenditure related to chair activity/motion is determined for the predetermined time interval. The user metabolic expenditure is then converted into desired outputs, such as sitting activity level, calories used from sitting activity, and equivalent steps. If none of these outputs change, in the next step the determined chair activity level is received for the next predetermined time interval. If any of these outputs changes during the most recent time interval, the outputs are updated and the displayed outputs are updated accordingly. At this point, the determined chair activity level is received for the next predetermined time interval.

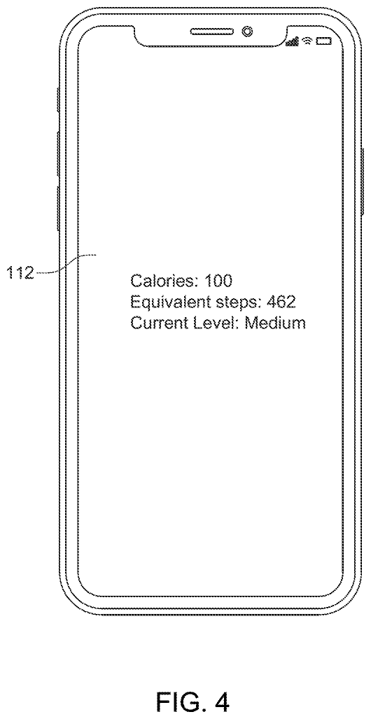

As shown in FIG. 4, a display 112 may be in communication with the processor to display, at the end of a sitting session or in real time, one or more of the active chair sitting activity outputs. These outputs may include total equivalent steps, total extra calories burned, current sitting activity level (e.g., low, medium, high). Display 112 may also be used to encourage user activity by displaying prompts or information linked to sensors 108, such as an icon that has its position on display 112 correlated with a current state of chair 100, such as a position and orientation of seat 102, based on data from sensors 108.

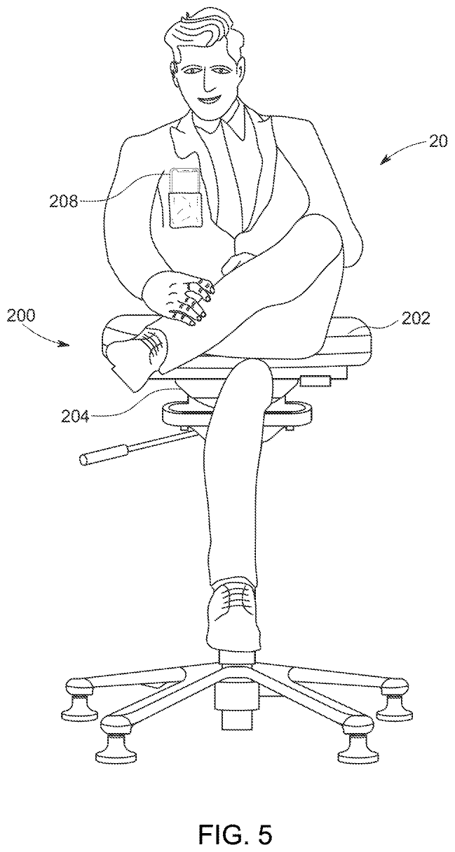

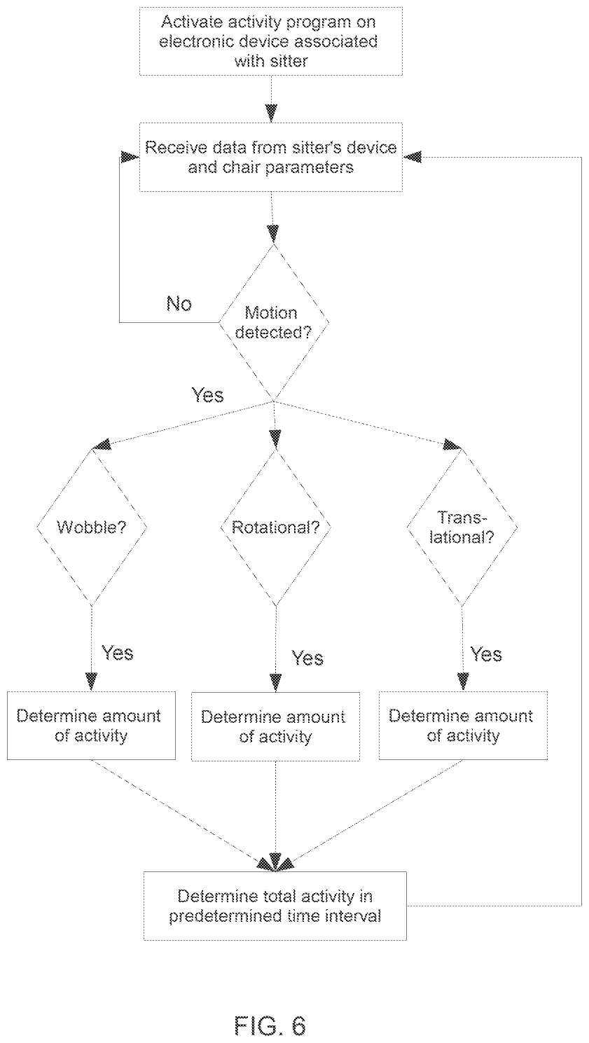

In another embodiment, the sensor/motion detector is located with the user/sitter, e.g., an accelerometer attached to the sitter or within an electronic device, such as a smart phone, held by a sitter, e.g., located in the sitter's pocket. FIG. 5 shows an active chair 200 having a wobble or rocker mechanism 204. A sitter 20 sits on a seat 202 of active chair 200 while carrying a device 208, such as a smart phone, in a pocket or otherwise attached to the sitter, that includes a motion detector such as an accelerometer and/or gyroscope. In a preferred embodiment, device 208 also includes an app or program that receives the detected motion data and determines the sitter's sitting activities from that data, as outlined in FIG. 6. Sitter and/or chair parameters may be entered into device 208 in order to facilitate more accurate determinations of activity and activity outputs (e.g., calories used for active sitting). The outputs may be transmitted via Bluetooth and displayed on the smart phone and/or on a device external to the smart phone such as a computer monitor and be updated at predetermined time intervals or based on certain changes in the outputs, e.g., changes in activity level or certain increases in calories used.

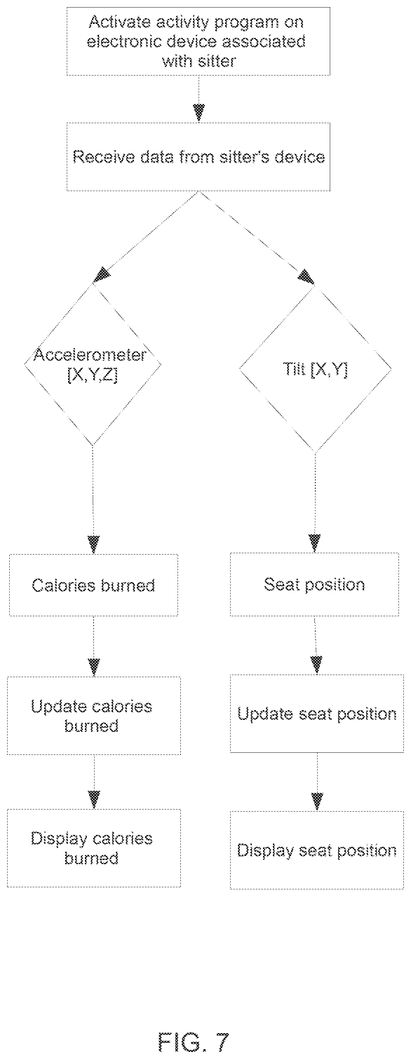

Another exemplary process for determining activity level and associated outputs is outlined in FIG. 7. When the program is activated by a user, data is received from the activity tracking sensors and motion detected by the accelerometer in any direction [X, Y, Z] is found as well as the tilt [X, Y]. From the motion data, calories burned are determined, updated, and displayed. From the tilt data, the current seat position is determined, updated and displayed. The seat position data may also be used to indicate a position on a monitor or within a window displaying an app in which an image or other image represents the current seat position with respect to the possible seat positions. This indication may be updated frequently, such as every 100 ms. In an embodiment, a maze or pattern could be shown that the sitter navigates with the seat position mark by controlling the position of the seat, or a target can be displayed that moves on the screen and the sitter causes the seat mark to follow the target by altering the position of the seat.

For any of the above embodiments, chair and user parameters are used to increase the accuracy of the determined outputs based on detected motions. For example, the type of chair will be associated with certain degrees and amounts of movement, which can inform the amount of energy associated with detected motions. Likewise, user parameters such as age and height allow these determinations to be further refined. In addition or in the alternative, detected motions may be associated with energy expenditure based on empirical analysis, e.g., associated with measured energy expenditure of similar users on similar chairs.

In addition to sensors attached to the active chair or the sitter and sensors incorporated into a smart phone or other device, the measuring, determining and/or displaying in accordance with the present invention may be integrated into a smartwatch or similar device.

The sitting activity outputs may be displayed on any of the above devices in real time, and cumulative data over a selected period of time (e.g., workday) may be provided, including via an activity versus time graph.

In an embodiment, an app allows a sitter to record and monitor a sitting session whenever they are sitting. Active sitting movements are tracked through internal smart phone sensors or an external sensor, such as a BlueTooth sensor, and converts raw acceleration to caloric burn. The app may also display the tilt of the sensor, representing the user's tilt on the chair, and includes a game that requires the user to tilt the chair in specific directions to follow a target.

The app includes stores, which may include an external sensor store, a motion store, a data store and a user store. The external sensor store accesses the external sensor data through an external sensor module and the motion store accesses the phone's acceleration and gyroscope data from internal sensors using a motion module. Both the motion store and the external sensor store contain a timer, and an increment function that is called at each timer increment when the user has started a sitting session. The increment may be any suitable amount, such as every 0.02 seconds, which provides a frequency of accelerometer data of 50 Hz. In operation, each data point is appended to an array, and at every minute, this array is passed through several functions to process and save the data, and the array is cleared for the next minute of data. Various functions convert the raw data to counts, which may be a unitless measure of acceleration.

The counts may be converted to calories by any suitable method including based on the potential energy method. The determined total calories burned for that minute are saved, may be displayed, and are accumulated during the session.

Alternatively, the total calories used for active sitting may be determined by relating active sitting motion counts per unit time to a basal metabolic rate (BMR) associated with the user. For this determination, for example, every 1 count per minute detected of sitting activity, the user's BMR is considered to increase by about 10%. Thus, the total calories burned may be found by determining the counts per minute, relating that value to an increase in BMR, and summing the total additional calories used for the time periods in a sitting session. In another alternative, calories for a sitting session may be determined based on the total distance traveled by the center of mass (of the sitter/chair) or based on the changes in the square of velocity of the sitter/chair about the tilt motion of the active chair. For example, the distance traveled may be determined based on the change of position of the sensor from one point in time to another. The velocity along that direction would be that distance divided by the time period between the points in time. Velocities may be determined in this manner for one or more directions (e.g., x axis and y axis) and the differences in determined velocities in a given direction or axis can be determined and squared and multiplied by one half the mass of the chair/sitter to yield an energy. The energies can be summed over all the measured velocity changes during a given sitting session to determine a total energy expenditure attributable to sitting motions during that sitting session.

The user's data is stored in the data store. This data may be used to create and maintain various variables representing the charts data and other user data.

Each of these stores are accessible in the app's views so that their data can be accessed and presented to the user. They may be initialized once at the launch of the app so that all views have access the same store, and therefore the same information.

In another aspect of the invention, all active sitting activities are monitored and accumulated based on the detection and distinctions of active sitting in comparison to other types of motion, such as walking or running. In this way, the user does not need to provide input to start a session when first sitting or stop a session when getting up from the active chair. For example, the program may distinguish (such as via machine learning) the type (e.g., based on direction, frequency, magnitude) of motion associated with active sitting motion from the type of motion associated with other movements by a user, such as walking. Motion detected by an accelerometer on a user while walking will have a different pattern than motion detected by the accelerometer on the user while engaged in active sitting.

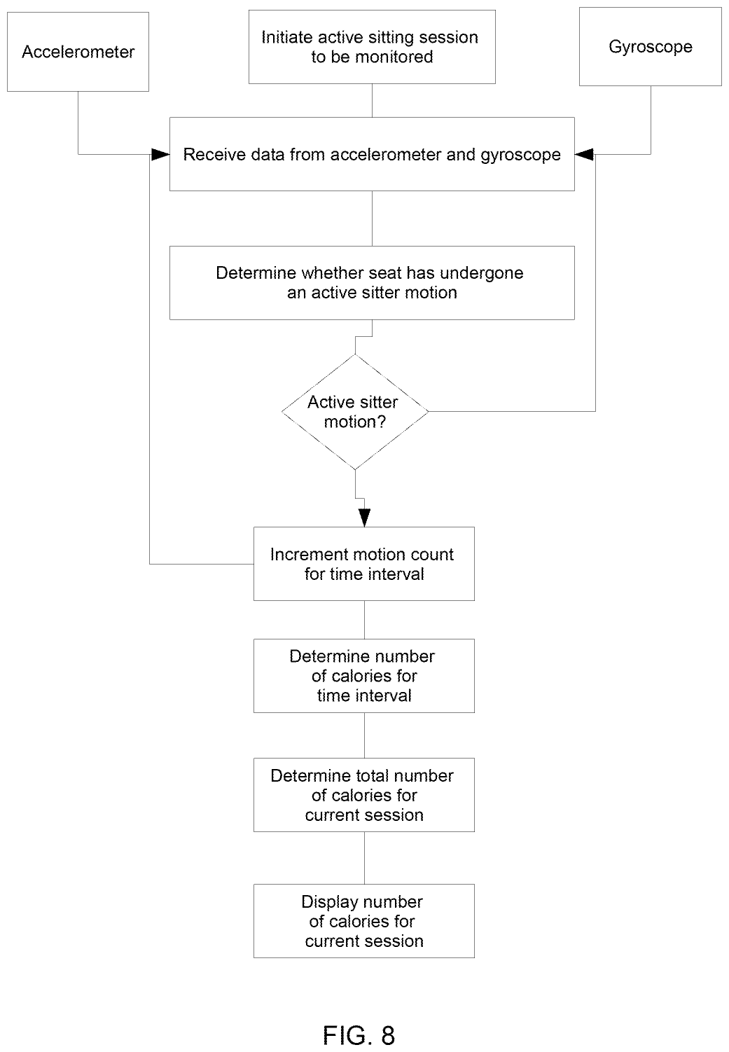

Another exemplary process for determining activity level and associated outputs is outlined in FIG. 8 in which motion information is obtained from a motion sensor such as an accelerometer configured to detect motion of an active chair when a user is seated on the active chair and orientation information is obtained from a gyroscope configured to detect a change in orientation of a seat of the active chair when the user is seated on the active chair. Based on the motion information and the orientation information, it is determined whether the seat has undergone an active sitter motion associated with the type of motion induced by the active chair when the user is seated in the active chair. During a predetermined interval, a motion count, or other measure of sitting activity, total is incremented whenever the seat has undergone the active sitter motion. Then, based on the sitting motion total accumulated during a user sitting session, a number of calories used by the user during the user sitting session is determined (via one or more of the techniques described above) and may be displayed on a display screen as well as tracked as part of session, daily, monthly, etc. totals, which may include comparing to goals, past averages, and equivalents to other activities such as walking or swimming.

In FIGS. 9A-9C, a display screen 300 is shown that includes a representation 308 of a current position of a seat of an active chair based on data from sensors on the active chair or on the sitter in the active chair that is translated in real time to representation 308 of a relative position/orientation of the active chair seat that is sent to and displayed on display screen 300. Display screen 300 may also include a target 304 that is displayed along with representation 308 in order to challenge the sitter to control the position/orientation of the seat through active sitter movements that result in repositioning of representation 308. In FIG. 9B, target 304 is moved such that representation 308 is no longer within target 304. In FIG. 9C, the sitter has engaged in motion that caused representation 308 to move back within target 304.

In addition, because good posture is also important to a healthy, pain free, back, in another embodiment the posture of the sitter is monitored and if poor posture is detected, an alert may be provided to the sitter. Further, a training session may be included that guides the sitter through an appropriate activity, such as series of pelvic postures and movements (e.g., a Pelvic Clock exercise) to promote both range of pelvic mobility and awareness of one's pelvic position.

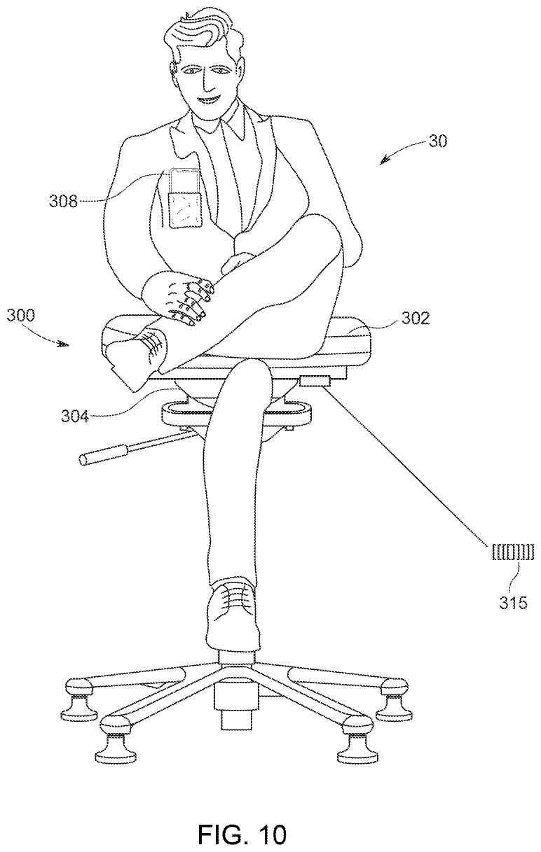

In an embodiment, a sensor is located on the sitter or the chair and configured to detect the sitter's posture. When a poor posture (such as sitting in a slumped position) is detected and sustained for more than a predetermined amount of time, e.g., one minute, an alert, such as a sound or text/image, is provided to the sitter. In an embodiment, the alert may persist or repeat until the sensor no longer detects the poor posture. To detect poor posture, the tilt of the seat or the sitter's pelvis may be monitored. As shown in FIG. 10, an active chair 300 has a wobble or rocker mechanism 304. A sitter 30 sits on a seat 302 of active chair 300 while a sensor 315, such as an accelerometer, or phone 308, is positioned to detect the tilt of the seat. Sensor 315 or phone 308 may include a motion detector such as an accelerometer and/or gyroscope. In a preferred embodiment, device 308 also includes an app or program that receives the detected motion data and determines the sitter's pelvis's or seat's tilt from that data,

If the tilt is greater than a threshold value, such as 10 degrees from horizontal, the siter's posture will be considered poor. If this condition, i.e., a detected tilt of 10 degrees or more, persists for a predetermined amount of time, such as 60 seconds, the system will determine that a sustained poor posture condition is occurring and send a signal to get the attention of the sitter. This signal or alert may persist or repeat until the detected poor posture is corrected, that is, the detected tilt of the seat becomes less than 10 degrees, for example.

When an improved posture is detected, an alert or signal may be provided to the sitter as feedback and to reinforce better posture.

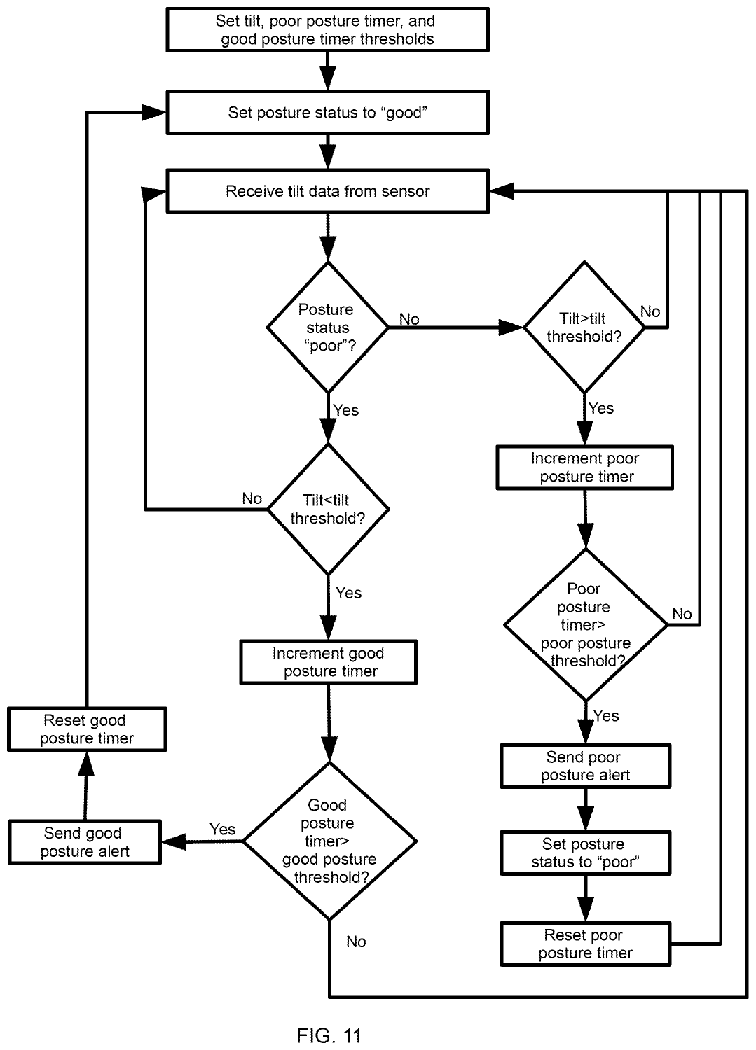

An overview of a posture detection and alert system is shown in FIG. 11. Threshold values are set for detected tilt degree, poor posture timer, i.e., the amount of time the sitter has been detected to be in a poor posture, and good posture timer, i.e., the amount of time the sitter has remained in a corrected good posture since poor posture was detected. As noted, the tilt degree threshold may be preferably set at 10 degrees from horizontal, but may be in suitable value. Likewise, the timer thresholds may be set at any amount of time, for example the poor posture timer may be set for 60 seconds and the good posture timer may be set at 1, 5, or 10 seconds. The default status is set to “good” as an initial condition.

A signal is received from a sensor located on a chair or sitter and configured to detect a tilt of the seat of the chair. If the posture status is not “poor,” the detected tilt is compared to a threshold tilt value and if the tilt is less than the threshold tilt value, the tilt is checked again at a predetermined interval, such as 5 seconds. If the detected tilt is greater than the threshold tilt value, a poor posture timer is incremented and the current poor posture timer value is compared to the poor timer threshold. If the poor posture timer value is not greater than the poor timer threshold, the tilt is checked again. If the over-tilting condition persists until the timer reaches a predetermined period, i.e., the poor posture timer value becomes greater than the poor timer threshold, an alert is sent to inform the sitter of the poor posture condition, the posture status is set to “poor”, and the poor posture timer is reset. At this point the tilt continues to be detected and the alert may persist until the tilt drops below the threshold value. When tilt data is received, the posture status is now “poor” and if the current tilt value is still greater than the tilt threshold, the next tilt data is received and processed the same way. If the detected tilt becomes less than the tilt threshold, the good posture timer is incremented. If the good posture timer is not greater than the good posture threshold, the next tilt data is received. If the good posture timer is greater than the good posture threshold, a good posture alert or message may be sent to the sitter, the good posture timer is reset, and the posture status is set to “good”.

In addition, a training aspect may also be included that assists sitters/users to improve posture generally by guiding them through a patterned series of movements designed to improve pelvic mobility and awareness. The pattern of movements for the guidance aspect may be any suitable series of movements, including the movements of a Pelvic Clock exercise in which the pelvis (or, in the upright position, the coccyx) at rest is envisioned to be at the center of an imaginary clock face, and one then tips the pelvis, slowly and with careful, deliberate attention, to the clock position indicated in the series. For example, when the movement value is 12, the user would move their pelvis or coccyx from the center of the clock to 12:00 (tip forward), then back to the center. A complete series of tips for this exercise could be as follows:
a=0,12,0
b=0,6,0
c=0,12,0,6,0
d=0,3,0
e=0,9,0
f=0,3,0,9,0
g=0,6,5,4,3,4,5,6
h=0,3,2,1,12,1,2,3
i=0,6,5,4,3,2,1,12
j=12,1,2,3,4,5,6,0,12
k=0,3,0,9,0
l=0,6,7,8,9,8,7,6
m=0,9,10,11,12,11,10,9
n=0,6,7,8,9,10,11,12,0,6,7,8,9,10,11,12,0, 6,7,8,9,10,11,12,0,6,7,8,9,10,11,12,0
o=12,11,10,9,8,7,6,0,12
p=0,3,0,9,0
q=0,6,0,12,0
r=0,12,11,10,9,8,7,6,5,4,3,2,1,12
s=0,1,2,3,4,5,6,7,8,9,10,11,12
t=0,12,0,6,0
u=0,3,0,9,0

Directions for such series of movements may be provided to the sitter/user in audio and/or visual form at a selected pace (e.g., about 15 minutes at 3 seconds per transition, or 10 minutes at 2 seconds per transition).

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for tracking a sitter's posture comprising:
an active sitting chair including a seat, a rocking mechanism, and a base;
a sensor configured to detect a tilt of the seat with respect to a horizontal axis; and
a device having a memory and a processor in communication with the sensor,
wherein:
the device is in communication with the sensor and the memory stores computer-executable instructions for causing the processor to:
receive data about the tilt of the seat from the sensor and determine a tilt value;
compare the tilt value to a tilt threshold; and
send an alert perceivable to a sitter on the active sitting chair when the tilt value is greater than the tilt threshold for a predetermined amount of time.

2. The system of claim 1, wherein the instructions cause the processor to increment a poor posture timer when the tilt value is greater than the tilt threshold.

3. The system of claim 2, wherein the instructions cause the processor to compare the predetermined amount of time to the poor posture timer and send the alert when the poor posture timer is greater than the predetermined amount of time.

4. The system of claim 3, wherein the instructions cause the processor to receive the data about the tilt every five seconds.

5. The system of claim 4, wherein the tilt threshold is 10 degrees and the predetermined amount of time is 60 seconds.

6. The system of claim 3, wherein the instructions cause the processor to receive, after the alert is sent, the data from the sensor, and to compare the tilt value to the tilt threshold.

7. The system of claim 6, wherein the instructions cause the processor to send a perceivable corrected posture alert when the tilt value is less than the tilt threshold for a second predetermined amount of time.

8. The system of claim 7, wherein the instructions cause the processor to increment a correct posture timer when the tilt value is less than the tilt threshold.

9. The system of claim 8, wherein the instructions cause the processor to compare the second predetermined amount of time to the correct posture timer and send the perceivable corrected posture alert when the corrected posture timer is greater than the second predetermined amount of time.

10. The system of claim 1, further including a display, wherein the alert is sent to the display.

11. The system of claim 10, wherein the alert includes a patterned series of movements designed to improve pelvic mobility and awareness of the sitter.

12. A method for tracking a sitter's posture comprising:
receiving data from a sensor configured to detect a tilt with respect to a horizontal axis of a seat of an active sitting chair having a rocking mechanism;
determining a tilt value from the data;
comparing the tilt value to a tilt threshold; and
sending an alert perceivable to a sitter on the active sitting chair when the tilt value is greater than the tilt threshold for a predetermined amount of time.

13. The method of claim 12, further including incrementing a poor posture timer when the tilt value is greater than the tilt threshold.

14. The method of claim 13, further including comparing the predetermined amount of time to the poor posture timer and sending the alert when the poor posture timer is greater than the predetermined amount of time.

15. The method of claim 14, further including receiving the data every five seconds.

16. The method of claim 15, wherein the tilt threshold is 10 degrees and the predetermined amount of time is 60 seconds.

17. The method of claim 12, further including receiving, after sending the alert, the data from the sensor, determining the tilt value, and comparing the tilt value to the tilt threshold.

18. The method of claim 17, further including sending a perceivable corrected posture alert when the tilt value is less than the tilt threshold for a second predetermined amount of time.

19. The method of claim 18, further including incrementing a correct posture timer when the tilt value is less than the tilt threshold.

20. The method of claim 19, further including comparing the second predetermined amount of time to the correct posture timer and sending the perceivable corrected posture alert when the corrected posture timer is greater than the second predetermined amount of time.

21. The method of claim 20, further including displaying on a screen viewable to a sitter on the active sitting chair, the alert.

22. The method of claim 21, wherein the alert includes a patterned series of movements designed to improve pelvic mobility and awareness of the sitter.
